(19) United States
(12) Patent Application Publication
(10) Pub. No.: US 2025/0060617 A1
(43) Pub. Date: Feb. 20, 2025

(54) LUMINESCENT CONTACT LENS

(71) Applicant: Ioan Corneliu DONIGA, MAINZ (DE)

(72) Inventor: Ioan Corneliu DONIGA, MAINZ (DE)

(21) Appl. No.: 18/725,723

(22) PCT Filed: Dec. 23, 2022
(86) PCT No.: PCT/EP2022/087751

§ 371 Date: Jun. 28, 2024

(30) Foreign Application Priority Data
Dec. 30, 2021 (DE) 102021215129.9

(51) Int. Cl. G02C 7/02; A61N 5/06; G02C 7/04

(52) U.S. Cl. CPC G02C 7/021; A61N 5/0618; G02C 7/049; A61N 2005/0648

(57) ABSTRACT

A contact lens is to be provided which can be used specifically for therapeutic purposes in the context of light therapy acting on the retina. For this purpose, the contact lens is designed as a luminescent contact lens with luminescent particles which, when viewed from above, has a plurality of segments which are provided with luminescent particles of different concentrations.

### CROSS-REFERENCE TO RELATED APPLICATION

This application is a national phase of International Application No. PCT/EP2022/087751, filed Dec. 23, 2022, which is hereby incorporated by reference in its entirety.

### FIELD

The disclosure relates to a luminescent contact lens.

### BACKGROUND

Colored or luminescent contact lenses are used for entertainment purposes or for aesthetic reasons to create iris colors that differ from nature. In the case of luminescent contact lenses, fluorescent dyes contained in the contact lens produce a short-lasting fluorescence when illuminated with UV light, which is perceived as a strong glow, particularly in dark surroundings.

It is also known that (visible) light has an influence on the circadian rhythm and that disorders of the serotonin-melatonin metabolism caused by a lack of light in particular can cause mental illness. The first systematic descriptions of autumn and winter depression date back to the beginning of the 19th century, including light therapy (winter tourism to southern climes). In the meantime, Seasonal Affective Disorder (SAD) has become one of the most important research topics of recent times, not least due to the pressure of a growing number of sufferers. The incidence varies depending on the country or latitude and averages around 5% of the population. The symptoms of autumn/winter depression are mainly characterized by a lack of energy and a depressive mood, albeit a milder one. In addition, there is daytime tiredness and an increased need for sleep as well as anxiety. Two thirds also show an increased appetite, especially a craving for carbohydrate-rich foods (pasta dishes, sweets).

Light therapy devices with high illuminance levels, for example, are used to reduce the lack of light. The light source is bright, white (fluorescent) light, which covers the entire spectrum of sunlight with the exception of the UV range, which is harmful to the retina. This can significantly improve well-being and performance within a few days. The devices require the person concerned to sit in front of the light-emitting device at a short distance for a certain period of time, as required, and to be exposed to the light. This is only possible if appropriate opportunities, e.g. at home after work, and time are available. The personal effort for the therapy increases even more if treatment is carried out clinically. This limits the usability of such devices in everyday life.

Although light therapy can achieve good results in the treatment of such diseases, the treatment is quite costly in terms of the light source required and the time needed for the treatment. It would therefore be desirable to use self-illuminating contact lenses, for example, which could be worn during everyday life and have a virtually imperceptible preventive or therapeutic effect. However, the known fluorescent contact lenses are unsuitable for this purpose due to the short lifespan of the fluorescent dyes and the low light intensity.

The object of this disclosure is therefore to provide a contact lens that can be used specifically for therapeutic purposes in the context of light therapy acting on the retina.

According to this disclosure, this object is achieved with the contact lens with the features of claim 1. The subclaims describe advantageous embodiments of this disclosure.

The basic idea according to this disclosure is to equip contact lenses with luminescent, preferably phosphorescent particles instead of fluorescent dyes, which have a purely aesthetic effect due to their short service life, and which produce a therapeutic effect for the wearer of such contact lenses when the luminophore has a longer service life.

In accordance with this disclosure, light therapy can be used much more simply and at the same time more widely. The inhibition threshold for the use of light of a suitable wavelength against winter depression or sleep disorders, or even just for physical “refreshment” against the onset of exhaustion, can thus be significantly reduced, whereby the same effect as with a light therapy device, but with much lower luminosity, can be achieved by placing the contact lens directly in front of the eye.

In accordance with this disclosure, a luminescent contact lens with phosphorescent particles is therefore proposed. In order to enable, in the sense of a therapeutic application, on the one hand a high effectiveness in the generation of the light directed into the eyes and at the same time a high tolerance, i.e. in particular avoiding overloading of the eye or excessive glare, the contact lens according to one aspect of this disclosure has a plurality of segments or sections, seen in plan view, which are provided with phosphorescent particles of different concentrations. By appropriately specifying the concentrations in different segments, the amount of light generated locally can be adjusted so that, for example, particularly sensitive areas of the eye or retina are only illuminated with little or possibly no light at all, whereas other, more suitable areas can be exposed to the light more intensively.

In particular and according to a preferred aspect of this disclosure, a central one of the segments provided for the pupil of the wearer has a lower concentration of luminescent particles than a neighboring region, and can particularly preferably also be kept completely free of luminescent particles. Furthermore, according to one aspect of this disclosure, it is provided that the luminescent contact lens has the phosphorescent particles exclusively in an area arranged above the pupil. Accordingly, in this preferred embodiment, the luminescent particles are contained exclusively in segments which, when worn, are provided for areas at least partially above the pupil of the wearer.

According to one aspect of this disclosure, a type of gradient loading of the contact lens with the luminescent particles can also be provided, in which the concentration of the particles decreases or increases more or less continuously along a predetermined path. In purely chemical terms during the manufacture of the contact lens, a distribution in concentration gradients can be implemented, for example, depending on the selected “polymerization dynamics”, as the particles will accumulate differently depending on their size after dispersion, depending on the selected curing and segmentation process.

In order to approximate such a gradient-like or continuous course of the concentration of the particles, according to one aspect of this disclosure it can also be provided that for a plurality of adjacent segments the concentration of luminescent particles increases continuously between two neighboring segments.

The luminescent particles are preferably designed as phosphorescent particles which, in a further advantageous embodiment, emit light in the wavelength range 400 nm≤λ≤550 nm, i.e. in the violet, blue and/or green spectral range.

In particular, it is proposed to use strontium aluminate (Sr₄AlO₁₄₂₅) doped with europium (Eu²⁺) and dysprosium (Dy³⁺) as phosphorescent particles. The phosphorescent particles can consist of pure strontium aluminate (Sr₄AlO₁₄₂₅) doped with europium (Eu²⁺) and dysprosium (Dy³⁺) or at least contain it. Strontium aluminate (Sr₄AlO₁₄₂₅) (CAS No. 76125-60-5) doped with europium (Eu²⁺) and dysprosium (Dy³⁺) is particularly suitable due to the increased luminous intensity resulting from the doping with europium in conjunction with dysprosium, whereby the phosphorescence can be further increased by adding silver. Strontium aluminate doped with europium (Eu²⁺) and dysprosium (Dy³⁺) (Sr₄AlO₁₄₂₅) exhibits an emission maximum of 485 nm, which is close to the absorption maximum of the melanopsin receptor of 484 nm. The doped strontium aluminate can be excited particularly in the longer wavelength UV-A range of approx. 365 nm, whereby selective excitation of the europium (2) center with the emission maximum of 485 nm is achieved.

On the one hand, the phosphorescent particles can have a hydrodynamic diameter of 20 to 140 μm and are therefore also optically perceptible without aids. In the present context, these particles can be processed very successfully and very effectively in the researched contact lens polymers and can then also be used in a measurably and visibly effective manner.

Alternatively, the phosphorescent particles may be nanoparticles, which most preferably have a hydrodynamic diameter of 105 nm to 460 nm. These are produced in particular by laser ablation. With this size, it can be ensured on the one hand that the particles do not primarily impair the optical properties of the contact lens, while at the same time the light emitted by these particles is therapeutically effective.

The luminescent contact lens is preferably a hydrogel or silicone hydrogel contact lens, in which a homogeneous distribution in the contact lens material can be achieved without washing out the luminophore. The phosphorescent particles are therefore preferably dispersed in the luminescent contact lens.

Finally, the use of strontium aluminate (Sr₄AlO₁₄₂₅) doped with europium (Eu²⁺) and dysprosium (Dy³⁺) according to this disclosure is proposed for the treatment of melatonin-associated diseases by means of light therapy, which is intended in particular for the treatment of winter depression. Strontium aluminate (Sr₄AlO₁₄₂₅) (CAS No. 76125-60-5) doped with europium (Eu²⁺) and dysprosium (Dy³⁺) is particularly suitable due to the increased luminous intensity resulting from doping with europium in conjunction with dyspropium, whereby the phosphorescence can be further increased by adding silver. Strontium aluminate doped with europium (Eu²⁺) and dysprosium (Dy³⁺) (Sr₄AlO₁₄₂₅) exhibits an emission maximum of 485 nm, which is close to the absorption maximum of the melanopsin receptor of 484 nm. The doped strontium aluminate can be excited particularly in the longer wavelength UV-A range of approx. 365 nm, whereby selective excitation of the europium (2) center with the emission maximum of 485 nm is achieved.

According to an aspect of this disclosure which is regarded as independently inventive, the charging of the luminescent contact lens can take place in an associated storage container for the contact lens. A storage container suitable for this purpose and provided in accordance with one aspect of this disclosure is known from WO 2019/202084 A1, the disclosure of which, in particular with regard to the energy supply of the contact lens and the charging of the embedded particles, is expressly included (“incorporation by reference”).

According to an aspect of this disclosure which is regarded as independently inventive, a matching ensemble of box or storage container on the one hand and lenses on the other hand is provided, wherein, in particular according to one aspect of this disclosure, a detection system can be provided which recognizes that lenses to be charged are in the container. For this purpose, a sensor for detecting the phosphorescence of the contact lens in the lens chambers (as a reaction to the irradiation in the chambers) can be provided in the storage container, for example. The radiation emitted by the contact lens can thereby be detected by sensors in the lens chamber, thereby confirming the presence of the lens in the chamber. For this purpose, according to one aspect of this disclosure, a specifically associated segment of the contact lens may be provided for a contact lens not otherwise intended for therapeutic purposes, for example in the form of a narrow outer luminous ring. In a contact lens of the type described above intended for therapeutic purposes, on the other hand, segments suitable for this purpose are provided anyway.

Since scintillation of the absorbed wavelength takes place in the contact lens itself, the light emitted by the contact lenses has a different wavelength than the UV light emitted in the storage container to charge the contact lens, so that it is comparatively easy for the sensors to reliably distinguish the light emitted by the storage container for charging from the light emitted by the contact lens.

### BRIEF DESCRIPTION OF THE DRAWINGS

An embodiment of this disclosure, in which the phosphorescent particles of strontium aluminate (Sr₄AlO₁₄₂₅) doped with europium (Eu²⁺) and dysprosium (Dy³⁺) were used, as shown in Table 1, is explained in more detail with reference to a drawing. It shows:

FIG. 1 is a schematic view of the basic process for manufacturing the contact lenses according to this disclosure,

FIG. 2 illustrates one contact lens each,

FIG. 3 is an optical microscope image of the cross-section of a contact lens,

FIG. 4 illustrates some luminescence excitation spectra,

FIG. 5 is an overview of the phosphorescence properties,

FIG. 6 is a schematic top view of a contact lens,

FIG. 7 illustrates the contact lens according to FIG. 6 in schematic representation in lateral view, and

FIG. 8 illustrates different variants of the contact lens according to FIG. 6, each in perspective view.

Identical parts are marked with the same reference signs in all figures.

### DETAILED DESCRIPTION

For the production of hydrogel and silicone hydrogel contact lenses, no significant adjustment of the relevant process parameters such as initiator concentration in the formulation, temperature profiles in the thermal copolymerization conditions and the polymerization process duration are required for the polymerization of the modified contact lens materials compared to the state of the art. This indicates that there are no strong inhibitor side effects due to the integration of the phosphorescent particles into the reactive monomer formulation. If the particle concentration is not much higher than about 1 wt %, the mechanical lens properties hardly change.

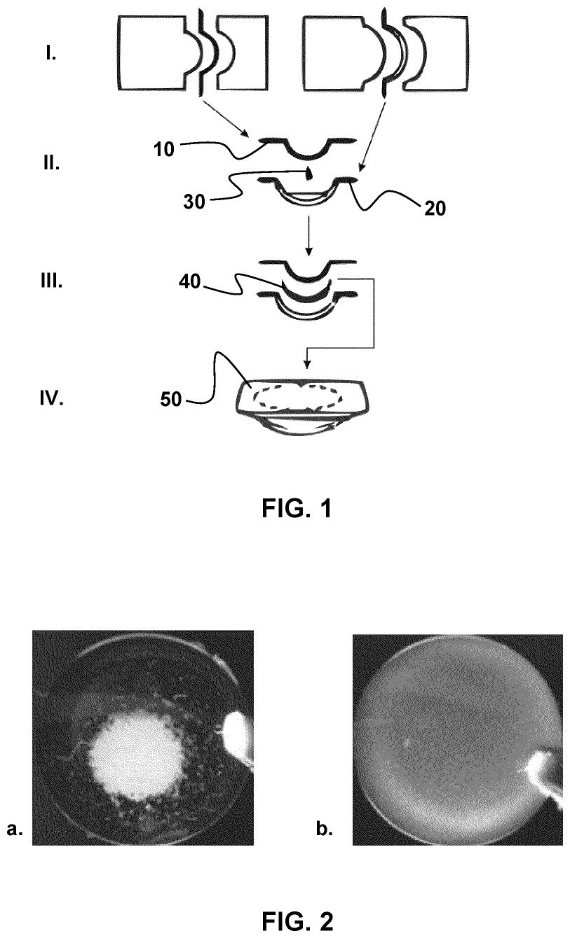

FIG. 1 shows a schematic view of the basic process for producing the contact lenses according to this disclosure. In a first step (I), molds 10, 20 are created for producing the contact lenses 40 to be manufactured, and in a second step (II), the polymer containing the phosphorescent particles is placed between them and cured. After demolding in a third step (III), the finished contact lens can be packaged in a fourth step (IV), for example in a blister pack.

In addition to the mechanical shape and strength properties of the resulting hydrogel contact lenses, the chemical-physical material properties were also investigated using different model formulations.

The stability and size of the measured values for surface wettability (contact angle), water absorption (water content) and oxygen permeability (Dk values) are particularly important for the successful physiological use of the medical device on the healthy eye. Using lens models with the very large particles PBG-6L (No. 1 in Table 1), among others, with a diameter of approx. 94 μm, the gas permeability for oxygen was determined as an example in the standardized test method according to ISO 18369-4:2017 and no significant difference was found at a particle weight fraction of 0.50% in pHEMA hydrogel lenses compared to simultaneously produced and measured pure pHEMA lenses without particle addition (approach IK234-62). Lens samples of the same center thickness were systematically compared and had the same Dk values.

This desired “inconspicuousness” was also found for the lens water content in measurements according to the normative basis ISO 18369-4:2017. Up to approx. 1 wt. %, no significant deviation of the water content due to the addition of nano- or macroparticles was found, with increases in the weight proportions of large particles, slightly reduced water absorption is gradually measurable, which, however, in the case of simple pHEMA polymers, only exceeds the normative tolerance of +/−2 wt. % above 3 wt. %. This can deviate slightly in individual cases for copolymers with a high water content.

These results are summarized in Table 2.

These very low effects found on the relevant chemical material properties mentioned here, among others, are indirectly a great advantage in stabilizing product biocompatibility despite the novel integration of chemically unbound and certainly higher weight proportions of phosphorescent particles of various sizes. Product risks for contact lenses of this type are therefore not obvious in the form of insufficient lens wetting, a greatly reduced water content or a critical undersupply of oxygen flux.

After hydration of the swellable, demolded lens polymer, the packaging in standard blisters with the buffered saline solution and steam sterilization by autoclaving without deformation of the lenses or particle extraction into the water-based solution was carried out in experiments on the present disclosure.

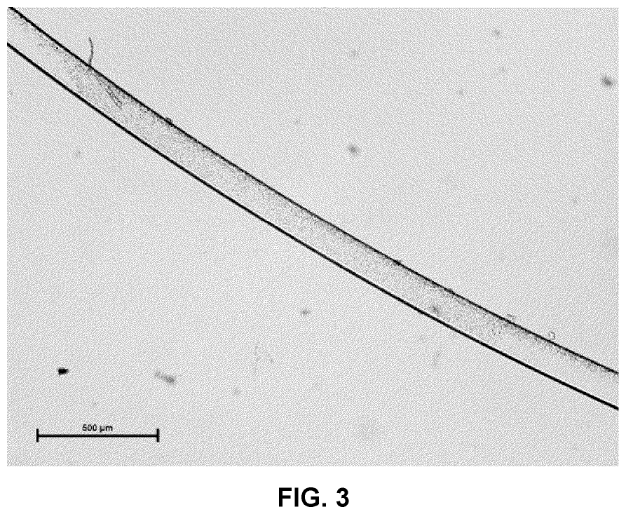

FIG. 2a shows a contact lens with an inhomogeneous distribution of phosphorescent particles obtained by sedimentation, whereas FIG. 2b shows a contact lens with a homogeneous distribution of phosphorescent particles. Other forms, in which an accumulation of the phosphorescent particles is achieved in certain sections of the contact lens, can be achieved using appropriate techniques (see also below).

FIG. 3 shows an optical microscope image of the cross-section of a particularly preferably designed contact lens according to this disclosure. In order to visualize the particle distribution in the finished, hydrogenated contact lens, microscopic images of cross-sectional contact lens samples were taken, the contact lens shown having been produced by a combination of a centrifugation step to increase the particle density and the subsequent thermal polymerization.

In general, however, a dispersion process was preferably used in the first step for modified soft contact lenses with specific, non-reactive inorganic particles. Here, the desired concentration of particles, specifically particles of strontium aluminate doped with europium (Eu²⁺) and dysprosium (Dy³⁺) (Sr₄AlO₁₄₂₅), was stirred into the transparent liquid monomer formulation at room temperature to achieve the best possible dispersion. After a longer stirring phase, this mixture was used as quickly as possible to fill the lens moulds and start the polymerization process. A short homogenization phase in advance with ultrasonic energy has a supporting effect. This mixture could already contain all formulation components, including the crosslinker components and the preferred initiator component(s). A fast reactive polymerization process from the field of casting (cast-moulding) was particularly suitable. However, if the polymerization processes take many hours, as is often required for the polymerization of large-area polymer intermediates (blanks, rod polymers, etc.), it becomes much more difficult to ensure homogeneous particle distribution in the material due to particle sedimentation.

The contact lens polymers with particle loading tested in the present studies were therefore obtained in a comparatively fast polymerization process of approx. 15 to max. 30 minutes at a maximum heat input of 97° C. The lenses were filled into polypropylene moulds consisting of two exactly matching mould halves. The lenses were filled into polypropylene molds consisting of two exactly matching mold halves. The dimensions of the molds were adjusted to the determined material source parameter in physiological buffered saline solution and to the desired contact lens geometry. This manufacturing process is the modern basis of today's hydrogel contact lenses in the field of so-called replacement products (daily and monthly disposable lenses). Building on the existing technology, homogeneous particle distributions have been achieved, particularly for the polyhydroxyethyl methacrylate copolymers mentioned above.

However, the possible applications are by no means limited to this, as copolymers with different hydrophilic comonomers to the HEMA could also be processed homogeneously with the particles. In the case of preferred particle compaction in the lens product, for better separation of loaded and unloaded lens sections, amphiphilic copolymers such as the so-called silicone hydrogels are preferred. The polar ratios in the liquid monomer mixture have a recognizable influence on the dispersibility and sedimentation speed and thus on the degree of particle distribution and their degree of agglomeration.

The resulting polymers could thus be transferred directly into the swelling liquid after demoulding from the PP moulds without post-processing (polishing etc.) and then autoclaved for sterilization (steam sterilization at 121° C./20 minutes) in the final packaging (cans or blisters).

No increased particle concentrations were measured in the storage solutions of the sterilized packaged contact lenses. As with larger particles, no migration of these particles (approx. 200 nm hydrodynamic diameter) during swelling and heat treatment was identified as a process risk. The particles did not significantly change the lens geometry and hardly or not at all changed the material properties. The water content was slightly (approx. ˜1 wt %) lower compared to equivalent polymers without particle loading. More important is the finding that the relevant oxygen permeability of these products remains practically unaffected.

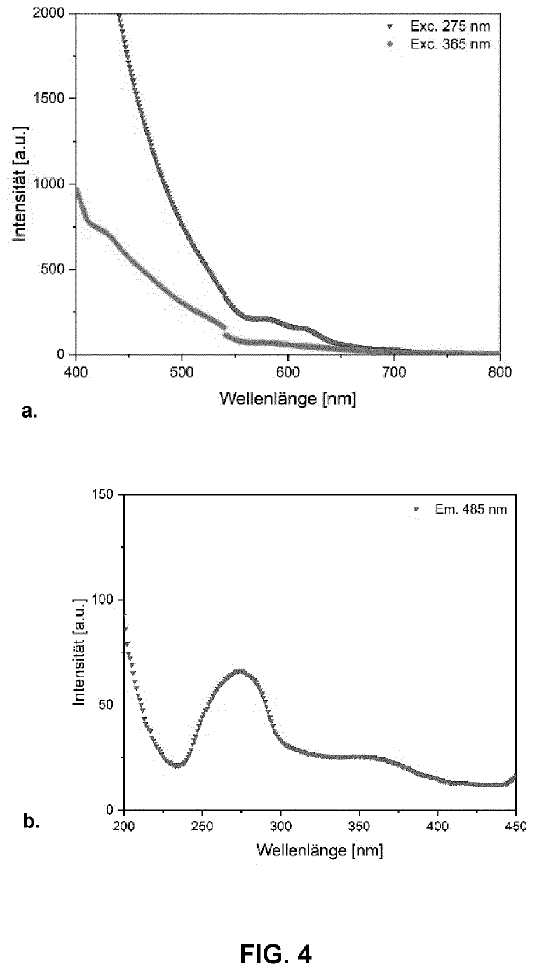

FIG. 4 shows the luminescence excitation spectra at λₑₘ=485 nm (a) and the emission spectra (b) after excitation at λ_{exc}=275 nm and λ_{exc}=365 nm of contact lenses modified with europium (Eu²⁺) and dysprosium (Dy³⁺) doped strontium aluminate (Sr₄AlO)·₁₄₂₅

It can be seen that when the particles are excited in the UV spectrum, phosphorescence with a maximum in the range of 485 nm can be achieved, whereby emitted light with this wavelength corresponds approximately to the absorption maximum of the melanopsin receptor at a wavelength of 484 nm.

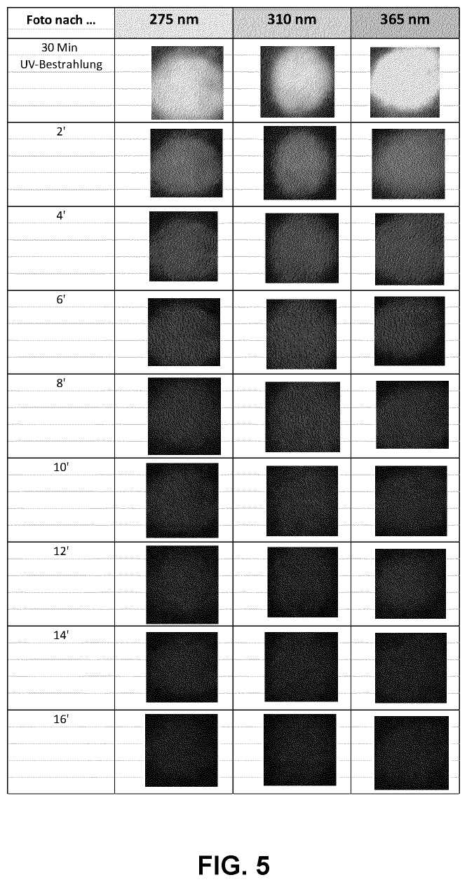

The hydrogel lenses according to this disclosure can be repeatedly activated with UV light as often as desired. For example, FIG. 5 shows that autoclaved Vitafilcon lenses loaded with 1% Realglow PBG-6L (94 μm) repeatedly form phosphorescence over approx. 10 to 30 minutes. Overall, phosphorescence usually lasts 15 to 25 min for all particles examined and at least 20 min for larger particles (>5 μm) in particular. It was clearly found that the luminous power depends on the quantity and even more clearly on the particle size. A compaction of the particles therefore has advantages on the phosphorescence, which can be utilized if the particles are located in defined sections outside the optical central zones. For this purpose, tools for producing a contact lens according to this disclosure must be specifically designed, e.g. squeezing devices that are filled with the lens monomer mixture in such a way that particle compaction occurs largely in the non-optical area before polymerization begins. The compaction of dispersed particles can also be controlled and intensified by process steps such as brief rotation or centrifugation.

The “decay behavior” of the light output of these lens models is both expected and desirable. Repeated activations are possible at will, i.e. continuous reuse in the wearing period of a monthly contact lens, for example, is easily applicable.

Cytotoxicity tests and eye irritation tests with luminescent contact lenses produced according to this disclosure provided no evidence of a toxic or irritating effect.

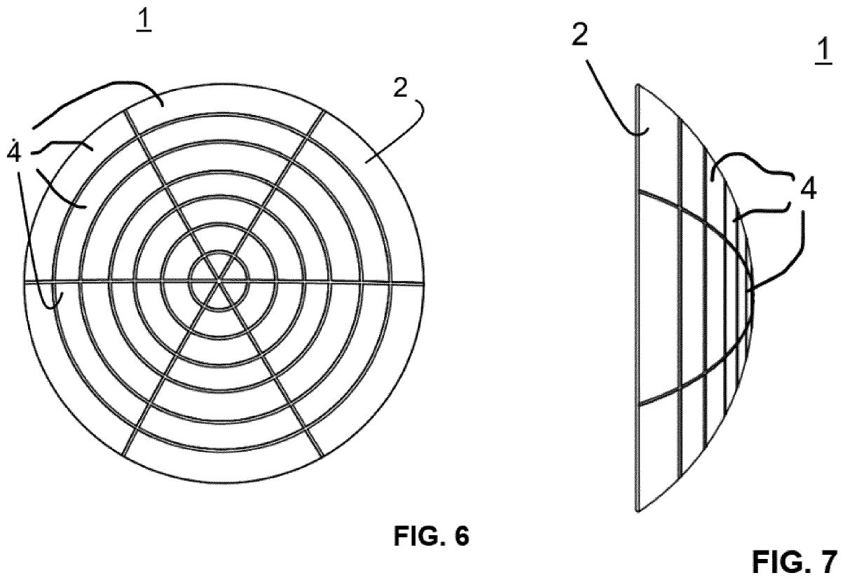

A segmented contact lens 1 according to one aspect of this disclosures shown schematically in plan view in FIG. 6. The contact lens 1 designed as a luminescent contact lens 1 is provided with phosphorescent particles as described above. It comprises a lens body 2, which is segmented and has a plurality of sections or segments 4 when viewed from above. These segments 4, which as shown in FIGS. 6, 7 can be arranged in a radial direction and/or in the form of circular segments lying next to one another, are provided with phosphorescent particles of different concentrations.

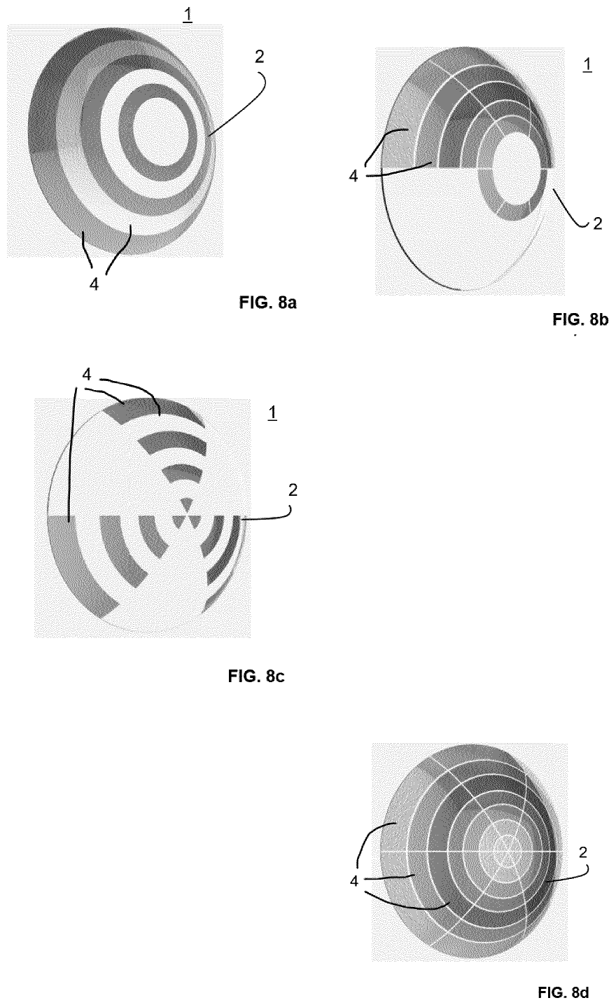

Some examples of possible designs of the contact lens 1 are shown in FIGS. 8a-d are shown in perspective view. The concentration of particles in the respective segment 4 is symbolized by the grey scale of the respective shading; a “darker” representation is intended to mean a comparatively higher concentration of particles. Light segments can also be completely free of particles.

FIG. 8a shows, for example, a luminescent contact lens 1 in which a central segment 4 intended for the pupil of the wearer is kept free of luminescent particles and thus has a lower concentration of luminescent particles than the neighboring segments 4. Otherwise, the particles in this embodiment example are arranged in the manner of ring-shaped segments 4. In the example according to FIG. 8b, in which the central segment 4 is also kept free of the particles, the luminescent particles are contained exclusively in segments 4, which are intended for areas at least partially above the pupil of the wearer when worn.

Depending on the intended purpose and mode of use, other constellations can also be selected. For example, FIG. 8c shows a variant in which the concentration in segments changes when viewed in the circumferential direction, and FIG. 8d shows a variant in which the concentration of luminescent particles increases continuously between two adjacent segments for a plurality of adjacent segments.

TABLE 1

| | | | | | Floureszenz | | Phosphoreszenz | |
|---|---|---|---|---|---|---|---|---|
| | | | | ~Größe (ist) | [nm] | | [nm] | |
| Nr. | Hersteller | Bezelchnung | Chem. Information | Bluewave [μm] | λ Ex. | λ Em. | λ Ex. | λ Em. |
| 1 | Realglow | PBG-6L | Sr₄Al₁₄O₂₅: Eu²⁺, Dy³⁺ | 94.13 (H2O): | 366 | 485 | 366 | 485 |
| | | | | 106.2 (iPrOH) | | | | |
| 2 | Realglow | PBG-6F | Sr₄Al₁₄O₂₅: Eu²⁺, Dy³⁺ | 40.2 | 366 | 485 | 366 | 485 |
| 3 | Kremer | Blau | (Sr, Ca)Al₂O₄: Eu²⁺, Dy³⁺ | 53.4 | 276.5 | 465 | 271 (360) | 465 |
| 4 | Araglow | 219 UV Blue | (Sr, Ca)Al₂O₄: Eu²⁺, Dy³⁺ | 80.99 | 276 | 465 | 274 | 465 |
| 5 | Araglow | 212 UV Green | SrAl₃O₄: Eu²⁺, Dy³⁺ | 89.29 | 268/317/363 | 513 | 270/315/364 | 513 |
| 6 | ColuxGlow | LB30W | Sr₄Al₁₄O₂₅: Eu²⁺, Dy³⁺ | 44.13 | (278)/363 | 486 | ~200 ggf< | 486 |
| 7 | ColuxGlow | OB30W | (Sr, Ca)Al₂O₄: Eu²⁺, Dy³⁺ | 47.17 | 276/(357.5) | 465 | 272 | 465 |
| 8 | ColuxGlow | BT2715S | (Sr, Ca)Al₂O₄: Eu²⁺, Dy³⁺ | 23.52 | 275.5/(357.5) | 465 | 271.5/(352.5) | 466 |
| 9 | Nightec | Blau-Grün Fein | Sr₄Al₁₄O₂₅: Eu²⁺, Dy³⁺ | 5.7 | (278.5)/360.5 | 485 | ~200 ggf< | 485 |
| 10 | Nightec | Blau-Grün Grob | Sr₄Al₁₄O₂₅: Eu²⁺, Dy³⁺ | 144.5 | (279)/364.5 | 485 | ~200 ggf< | 485 |

TABLE 2

| | Examination IK232-29: (mono | | | |
|---|---|---|---|---|
| | lymerized in PHEMA/MA) Parti | | | |
| | kel: NighTec blue-green- | | | |
| | fine(5.7 μm) | | | |
| | 0% | 1% | 3% | 5% |
| grav. H₂O content determination in % | 50.46 | 50.42 | 50.25 | 49.78 |

| | | |
|---|---|---|
| Contact angle determination (wetting) | 29° | Water |
| | | droplets melt |

### LIST OF REFERENCE SYMBOLS

1 Contact lens
2 Lens body
4 Segment

1. A luminescent contact lens, comprising luminescent particles which, when viewed from above, has a plurality of segments which are provided with luminescent particles of different concentrations.

2. The luminescent contact lens according to claim 1, wherein a central one of the segments provided for a pupil of a wearer has a lower concentration of luminescent particles than a neighboring region.

3. The luminescent contact lens according to claim 1 or 2, in which the luminescent particles are contained exclusively in segments which, when worn, are intended for areas at least partially above the pupil of the wearer.

4. The luminescent contact lens according to claim 3, wherein for a plurality of adjacent segments the concentration of luminescent particles between two adjacent segments increases continuously.

5. The luminescent contact lens according to claim 1, wherein phosphorescent particles are provided as luminescent particles.

6. The luminescent contact lens according to claim 1, wherein the luminescent particles emit light in the wavelength range 400 nm≤λ≤550 nm.

7. The luminescent contact lens according to claim 1, wherein the luminescent particles comprise strontium aluminate (Sr4Al14O25) doped with europium (Eu2+) and dysprosium (Dy3+) or are formed therefrom.

8. The luminescent contact lens according to claim 1, wherein the luminescent particles have a hydrodynamic diameter of 20 to 140 μm.

9. The luminescent contact lens according to claim 1, wherein the luminescent particles are nanoparticles.

10. The luminescent contact lens according to claim 9, wherein the nanoparticles have a hydrodynamic diameter of 105 nm to 460 nm.

11. The luminescent contact lens (1) according to claim 1, wherein the luminescent contact lens is a hydrogel or silicone hydrogel contact lens.

12. The luminescent contact lens according to claim 1, wherein the luminescent particles are dispersed in the luminescent contact lens.

13. A method for the treatment of melatonin-associated diseases with light therapy, comprising using of strontium aluminate (Sr4Al14O25) doped with europium (Eu2+) and dysprosium (Dy3+) in a contact lens.

14. A method for the treatment of winter depression, comprising using a contact lens according to claim 1.

15. The luminescent contact lens according to claim 2, wherein the central one of the segments provided for the pupil of the wearer is free of luminescent particles.

16. The luminescent contact lens according to claim 6, wherein the luminescent particles are phosphorescent particles.

17. The luminescent contact lens according to claim 7, wherein the luminescent particles are phosphorescent particles.

18. The luminescent contact lens according to claim 8, wherein the luminescent particles are phosphorescent particles.

19. The luminescent contact lens according to claim 9, wherein the luminescent particles are phosphorescent particles.

20. The luminescent contact lens according to claim 12, wherein the luminescent particles are phosphorescent particles.
